(19) United States
(12) Patent Application Publication
(10) Pub. No.: US 2020/0306187 A1
(43) Pub. Date: Oct. 1, 2020

(54) Composition of Athletic and Fitness Supplements, Method for Production and Method for Pulmonary Administration of Same

(71) Applicant: V Rush Vapes LLC, East Haven, CT (US)

(72) Inventors: Jason Thomas Bandy, East Haven, CT (US); Shun Hang Chan, New Haven, CT (US)

(21) Appl. No.: 16/833,593

(22) Filed: Mar. 28, 2020

Related U.S. Application Data
(60) Provisional application No. 62/825,869, filed on Mar. 29, 2019.

(51) Int. Cl. A61K 9/12; A61K 9/00; A61K 31/525; A61K 31/5025; A61K 31/10; A61K 31/17; A61K 36/752; A61K 36/21; A61K 47/10; A61M 15/00; A61M 15/06

(52) U.S. Cl. CPC A61K 9/12; A61K 9/0073; A61K 31/525; A61K 31/5025; A61K 31/10; A61M 15/009; A61K 36/752; A61K 36/21; A61K 47/10; A61M 15/003; A61M 15/06; A61K 31/17

(57) ABSTRACT

Disclosed is a performance-enhancing supplement for pulmonary administration wherein the supplement consists of a base e-liquid and a selected combination of supplement ingredients. The base liquid is selected from vegetable glycerin or propylene glycol, and the combination of ingredients is selected from at least three of pineapple lime extract, riboflavin, L-tyrosine, L-theanine, taurine, caffeine, agmatine sulfate, citrulline malate, and beetroot. The resulting performance-enhancing supplement is provided in a standard vape cartridge for use in an electronic cigarette device.

### CROSS REFERENCE TO RELATED APPLICATION

The present non-provisional application claims priority to provisional application Ser. No. 62/825,869, which was filed Mar. 29, 2019, entitled “Apparatus and Method for the Administration of Athletic and Fitness Supplements,” the disclosure of which is incorporated herein by reference.

### FIELD OF THE DISCLOSURE

This disclosure relates to pulmonary administration of athletic and/or fitness supplements to subjects engaged in rigorous exercise or fitness training, with a goal of enhancing the sports performance of those subjects. For example, athletic and/or fitness supplements may be vaporized using vaporizers, sublimators, vape pens, atomizers, battery-powered electronic-cigarette (e-cigarette) systems, and other similar devices into inhalable form, thereby enabling the systemic delivery of supplements via the airways into the bloodstream. This disclosure is applicable to the pulmonary administration of supplements before, during, and after exercise. The disclosure provides supplement formulations consisting of multiple ingredients that in combination promote energy, endurance, and mental focus to support enhanced athletic performance. Enhanced athletic performance may mean a qualitative perception by the individual athlete of having greater physical strength or better focus or more energy during training, or a quantitative factor such as increased muscular contractions and greater energy output, i.e., being able to lift heavier objects, run farther, or swim faster. Also disclosed are methods of administering other health and/or well-being related supplements to subjects using a vape-delivery system.

### BACKGROUND OF THE DISCLOSURE

Conventionally, sports supplements, especially the ones used before, during and after exercise (also known as pre-, intra- and post-workout supplements), are administered orally as liquids, capsules, tablets, or in powdered form. Oral administration of these sports supplements, however, has been reported by many to cause unwanted side effects, including gas or bloating, and digestive discomfort and stress, hindering optimization of athletic performance during and after the administration, and outweighing the benefits brought about by the sports supplement. In addition to such side effects, oral administration also varies amongst individuals and requires considerable time for digestion before the ingredient molecules are properly metabolized and absorbed into the body. Such delay in the onset of action poses inconvenience for athletes to plan their training or exercise schedule relative to the timing of supplements administration. Lastly, the metabolizing action of orally delivered supplements by the digestive system greatly reduces their concentration and bioavailability, hence requiring a higher dosage to achieve a desired effect.

An example of inhalation therapy for physical activity is disclosed in U.S. patent application Ser. No. 15/353,981, titled “Electronic Vapor Device Enabled Exercise System” by John Cameron, the content of which is incorporated herein by reference. This system is directed to determining and implementing a workout and vaping program based on a preexisting user profile. Yet this system has the disadvantage of requiring the use of costly and bulky computing and networking equipment and is limited in application to only a small set of exercise equipment. Moreover, the disclosure is silent regarding the composition and method of production of the fluid formulations to be aerosolized. The disclosure is similarly silent regarding the pre-workout and post-workout (recovery and stress relief) administration of aerosolized matter.

### BRIEF SUMMARY OF THE DISCLOSURE

In order to overcome and mitigate the deficiencies noted above with conventional oral administration of sports supplements, this disclosure presents novel pulmonary-delivered sports supplements. A feature of the present disclosure is the use of aerosolization technology to create inhalable forms of athletic supplements that can be delivered via pulmonary administration, bypassing a need for traditional oral administration. While other inhalable pharmaceutical products exist, the formulations disclosed herein are distinguished in their application and design as well as the content they deliver using a pulmonary route. In addition to the method of delivery, further novelty stems, in part, from multi-ingredient formulations that combine the benefits of different sports supplements to provide a synergistic enhancement in sports performance from increased energy, muscle power and endurance, as well as enhanced alertness. To date, there exist products that deliver alertness enhancement supplements via the pulmonary route, such as caffeine vape cartridges, for example. However, the design and formulation of these products based upon a single or dual ingredient recipe lacks the synergistic design and complexity of the multi-ingredient (mixture of a plurality of sport supplement ingredients and at least one base e-cigarette liquid (e-liquid) ingredient) formulations described herein.

As used herein, terms such as “enhanced athletic performance” or “performance enhancing” may refer to a qualitative perception by the individual athlete of having greater physical strength or better focus or more energy during training or to a quantitative factor such as increased muscular contractions and greater energy output, i.e., the ability to lift heavier objects, run farther, or swim faster than when not having taken the supplement or formulation.

### BRIEF DESCRIPTION OF THE DRAWINGS

The features and advantages of this disclosure will be more fully understood with reference to the following, more detailed description, when taken in conjunction with the accompanying figures.

FIG. 1 illustratively depicts an e-cigarette used to aerosolize an e-liquid and administer the resulting aerosol to a subject in accordance with the disclosure.

FIG. 2 is a flowchart that illustrates the steps in producing an embodiment of the present invention.

### DETAILED DESCRIPTION OF THE INVENTION

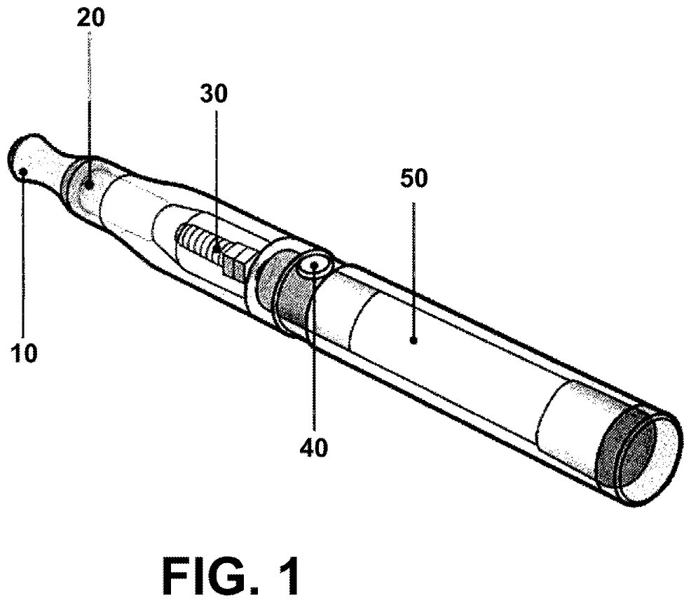

FIG. 1 shows an exemplary e-cigarette device that includes a mouthpiece 10 through which a user inhales an aerosol, a storage tank (cartridge) 20 that holds an e-liquid to be atomized (aerosolized), an atomizer 30 that heats and atomizes (aerosolizes) the e-liquid, a microprocessor (not shown), a control mechanism 40 which may be configured as a push-button and that is used to turn the e-cigarette on/off and for other functions, and a power source 50, typically a battery.

The cartridge 20 that holds the e-liquid may have an industry-standard 510 thread connection (10 threads equally spaced apart at 0.5 mm per thread) for compatibility with a wide range of e-cigarette devices. The e-liquid contained within the cartridge 20 may be formulated as a combination of a base e-liquid, such as vegetable glycerin (VG) or propylene glycol (PG), and a mixture of selected supplement ingredients to provide the desired effect and dosage.

In the following embodiments, ingredients are selected and blended together to produce formulations in which the effects of individual ingredients combine synergistically to enhance the overall beneficial effect of the formulation, while negating adverse side effects and allowing more moderate dosages. These ingredients would not produce the same effects if administered individually.

### Example 1: Pre- and Intra-Workout Formulation

In this embodiment, any combination of base solvent oils, such as VG or PG, and three or more of the following ingredients may be used to make a formulation. While any combination of three or more of the ingredients may be utilized, it is preferred that all ingredients be components of the composition. Ingredient percentages listed below are in volume percent.

| Ingredient | Range | Exemplary Formulation |
|---|---|---|
| VG (Vegetable Glycerin) | 45%-99% | 50% |
| Palm/Coconut Based | | |
| PG (Propylene Glycol) | 40%-99% | 46.28% |
| Pineapple Lime Extract | 0.5%-3% | 1% |
| Riboflavin | 0.05%-3% | 0.09% |
| L-Tyrosine | 0.1%-0.6% | 0.19% |
| L-Theanine | 0.1%-0.6% | 0.19% |
| Taurine | 0.3%-1.2% | 0.47% |
| Caffeine | 0.3%-1% | 0.38% |
| Agmatine Sulfate | 0.05%-0.3% | 0.09% |
| Citrulline Malate | 0.1%-0.6% | 0.19% |
| Beetroot | 0.05%-0.3% | 0.09% |

Caffeine, L-theanine and taurine, when administered together, provide a synergistic benefit to the user. As effective as caffeine can be in boosting one's energy level, it can also come with some drawbacks. Administering too much caffeine can easily lead to jitters and a characteristic energy crash once the caffeine has worn off. Mismanaging one's caffeine intake can also cause sleep issues, and lead to a heightened tolerance to caffeine, forcing one to consume increasingly greater dosage levels to sustain similar effects. L-theanine, however, when administered together with caffeine, can take the edge off of caffeine. The potential inhibitory nature and calming effects of theanine can nullify the jittery muscles, hyperactivity, and restlessness often associated with ingesting too much caffeine. Studies have shown that caffeine alone improves accuracy on attention-switching tasks, but that the combination of both caffeine and L-theanine improved both the speed and accuracy of performance at the same tasks, and reduced susceptibility to distracting information during memory tasks. Additional studies have shown that adding taurine to a caffeine and L-theanine mixture can provide the benefit of increased endurance. The foregoing describes an example of the synergistic benefits provided from three ingredients of the embodiment. Additional synergistic benefits that enhance athletic performance may be provided from the other ingredients.

The present disclosure relates not only to the formulation of and pulmonary administration of sports supplements, but also includes other formulations that provide ancillary and synergistic benefits thereto. Additional embodiments, such as a formulation to promote restful sleep and recovery, useful after strenuous training days, and a formulation to relieve stress and anxiety, which are common problems that could affect athletic performance and could build up from the strenuous trainings, for example, are listed below.

### Example 2: Sleep/Recovery Formulation

The following ingredients work synergistically to provide a more restful, deep and recovered sleep. The ingredients purposely blended together to provide different aspects to the sleep process including REM sleep. The resulting embodiment helps to relax the body, calm the central nervous system, and facilitate falling asleep quicker.

In this embodiment, any combination of base solvent oils, such as VG or PG, and three or more of the following ingredients may be used to make a formulation. While any combination of three or more of the ingredients may be utilized, it is preferred that all ingredients be components of the composition. Ingredient percentages listed below are in volume percent.

| Ingredient | Range | Exemplary Formulation |
|---|---|---|
| VG (Vegetable Glycerin) | 45%-99% | 50% |
| Palm/Coconut Based | | |
| PG (Propylene Glycol) | 40%-99% | 46.28% |
| 5HTP | 0.2%-1% | 0.38% |
| L-Theanine | 0.25%-1.5% | 0.57% |
| Melatonin | 0.005%-0.05% | 0.01% |
| Rutaecarpine | 0.1%-0.6% | 0.19% |
| Gamma Aminobutyric | 0.2%-1.2% | 0.38% |
| Acid (GABA) | | |
| Chamomile Flower Extract | 0.4%-2.4% | 0.76% |
| (matricaria recutita) | | |
| Tryptophan | 1%-6% | 1.9% |
| Huperzine A | 0.15%-1% | 0.29% |
| Evodiamine | 0.005%-0.05% | 0.01% |
| L-Ornithine | 0.4%-2.5% | 0.76% |
| Tart Cherry Extract | 0.4%-2.5% | 0.76% |
| Valerian Root | 0.85%-5.5% | 1.71% |
| Lavender Extract | 0.15%-1% | 0.3% |
| Passion Flower (passiflora | 0.4%-2.4% | 0.76% |
| incarnata) | | |
| Mucuna Pruriens L-Dopa | 0.5%-3% | 0.95% |
| Phosphatidylserine | 0.05%-0.3% | 0.095% |
| Reishi/Lingzhi | 0.1%-0.5% | 0.15% |

In this formulation, the ingredients of GABA, L-theanine, and melatonin provide synergistic sleep-enhancing benefits. The administering of a mixture of GABA and L-theanine has been found to decrease sleep latency (length of time to fall asleep) and increase sleep duration as compared to GABA or L-theanine alone. Moreover, the addition of melatonin enhances the effects of falling asleep faster while Gaba and L-theanine work in conjunction to keep the body relaxed and remain asleep. The foregoing describes an example of the synergistic benefits provided from three ingredients of the embodiment. Additional synergistic benefits that enhance athletic performance may be provided from the other ingredients.

### Example 3: Stress/Anxiety Formulation

The following ingredients work synergistically together to promote a calming of the mind and body. The embodiment helps to reduce stress, anxiety, depression, and to reduce central nervous system disruption due to anxiety and other stressors. Additional benefits are improved focus, cognition and energy. The combination of ingredients disclosed below creates a multi-beneficial effect to combat stress, anxiety and depression naturally.

In this embodiment, any combination of base solvent oils, such as VG or PG, and three or more of the following ingredients may be used to make a formulation. While any combination of three or more of the ingredients may be utilized, it is preferred that all ingredients be components of the composition. Ingredient percentages listed below are in volume percent.

| Ingredient | Range | Exemplary Formulation |
|---|---|---|
| VG (Vegetable Glycerin) | 45%-99% | 50% |
| Palm/Coconut Based | | |
| PG (Propylene Glycol) | 40%-99% | 46.28% |
| Gamma Aminobutyric | 0.2%-1.2% | 0.38% |
| Acid (GABA) | | |
| Chamomile Flower Extract | 0.4%-2.4% | 0.76% |
| (matricaria recutita) | | |
| Passion Flower (passiflora | 0.4%-2.4% | 0.76% |
| incarnata) | | |
| Bacopa Monnieri | 0.25%-1.5% | 0.57% |
| L-Phenylalanine | 0.2%-1.2% | 0.38% |
| L-Theanine | 0.25%-1.5% | 0.57% |
| Lemon Balm | 0.25%-1.5% | 0.57% |
| Gotu Kola | 0.5%-3% | 0.95% |
| Aniracetam | 0.1%-0.6% | 0.19% |
| Valerian Root | 1%-6% | 1.9% |
| Phenibut | 1%-6% | 1.9% |
| Alpha-GPC | 1.1%-7% | 2.28% |
| Ashwagandha (KSM-66) | 0.4%-2.4% | 0.76% |
| Rhodiola Rosea | 0.5%-3.5% | 1.14% |
| PQQ | 0.02%-0.12% | 0.038% |
| Noopept | 0.015%-0.1% | 0.029% |
| Cat's Claw | 0.5%-3% | 0.95% |
| Oat Straw | 1.1%-7% | 2.28% |

In this formulation, ashwagandha, Rhodiola rosea, and L-theanine provide synergistic stress-reducing benefits. Aswagandha and Rhodiola rosea are both adaptogenic herbs that work on the entire body and help it adapt to, and to function optimally in response to, internal and external stresses. Aswagandha and Rhodiola rosea regulate hormone levels and help the whole body function in a peak state and are useful to treat a wide variety of conditions. Moreover, the administration of these two ingredients in combination creates a synergistic effect that enhances the power and effectiveness of both compared to when administered individually. L-theanine may be added to this combination to additionally provide central nervous system (CNS) relief. The synergistic effect among ashwagandha, Rhodiola rosea, and L-theanine will ensure that the CNS is relaxed while any negative side effects of the energy enhancing effect of the Rhodiola rosea will be mitigated by the calming effect of the L-theanine.

Specifically, the administering of ashwagandha, Rhodiola rosea and L-theanine, taken together in combination, provides the following benefits:
Increased energy—greater physical and mental energy and a more controlled and pleasant energy boost than from caffeine
Improved mental focus—a great study aid for tests and exams
Reduced stress and anxiety—Improved mood and sense of well-being
Reduced depression
Aphrodisiac—increased sex drive and performance in both men and women
Enhanced athletic performance—may be administered before or during exercise or a sporting event to improve stamina and performance
Decreased CNS response to anxiety and stress, while calming the receptors and brain for a relaxed feeling
   The foregoing describes an example of the synergistic benefits provided from three ingredients of the embodiment. Additional synergistic benefits that enhance athletic performance may be provided from the other ingredients.

Additional embodiments may include one or more of the following supplement ingredients: tart cherry extract, L-carnitine, 5-HTP, L-theanine, melatonin, magnesium, zinc, L-glycine, rutaecarpine, gamma aminobutyric acid (GABA), CBD, chamomile flower extract (Matricaria recutita), lavender extract, passion flower (Passiflora incarnata), bacopa monnieri, lemon balm, tryptophan, huperzine A, gotu kola, aniracetam, quercetin, tribulus terrestris, vitamin C, vitamin E, coenzyme Q10, dehydroepiandrosterone (DHEA), curcumin, palmitoylethanolamide, Mucuna pruriens L-dopa, L-phenylalanine, L-ornithine, valerian root, phenibut, alpha-GPC, ashwagandha (KSM-66), evodiamine, phosphatidylserine, DMAE, hops strobile, lemon balm leaf, bacopa monnieri, Rhodiola rosea, sulbutiamine, alpha-lipoic acid, choline, PQQ, vinpocetine, pine bark, reishi/lingzhi, cordyceps, lion's mane, chaga, shiitake, pterostilbene, resveratrol, uridine monophosphate, theobromine, ginko biloba leaf, sulbutiamine, noopept, cat's claw, oat straw, L-norvaline, hordenine, synepherine, alcar, theobromine, elderberry, and zinc.

The method disclosed herein of administering sports-related supplements via the pulmonary route offers a noninvasive route to deliver athletic/sports supplements almost directly into the blood stream. According to this method, a therapeutic dose of a supplement is aerosolized, preferably by heating with an atomizer of an e-cigarette, and then administered via pulmonary administration to an athlete, before, during, or after a workout. This method of supplement administration: (1) bypasses the digestive system and hence avoids posing any digestive discomfort and stress; (2) offers rapid onset of action as the aerosolized molecules can be absorbed and delivered to the bloodstreams within seconds during inhalation; (3) provides improved bioavailability over orally delivered supplements since no additional digestion and metabolizing is involved, and hence achieves a more dose-effective administration of the intended supplements. Significant improvements in the ease of use, safety and power output of vaporizers, sublimators, vape pens, atomizers, and battery-powered e-cigarettes systems have been achieved over the years, and embodiments of the present disclosure may be adapted for usage with such devices to enhance the intuitiveness and user friendliness for athlete users, and also to realize greater accessibility and a more cost-effective design.

The method disclosed herein of the pulmonary administration of the sports-related supplements generally begins to produce beneficial effects within about 5 to 10 minutes from the time it is administered. A formulation intended for a pre-workout benefit will preferably be administered 5-15 minutes before exercising. A formulation intended for an intra-workout benefit may be administered at any time during the training session, and a formulation intended for a post-workout benefit may be administered as soon as immediately after the conclusion of the training session.

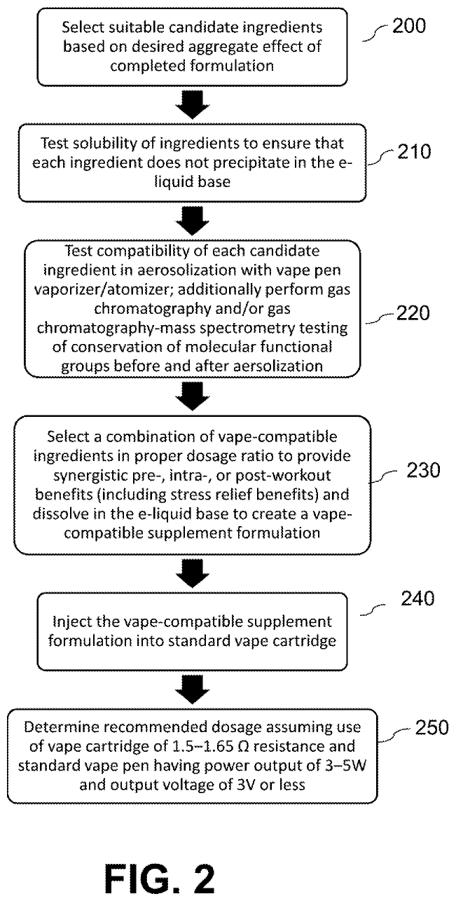

In accordance with an embodiment, disclosed is a method for producing a formulation of a vape for pre-workout delivery. This method is described below with reference to steps illustrated in FIG. 2. Pre-workout, in the fitness supplement industry, refers to any supplement (usually a powder drink mix) that is intended to increase energy levels, muscle power or endurance and mental focus during a workout if consumed prior beforehand. The disclosed method of producing a vape formulation is not limited to pre-workout formulations and may also be applied to produce other types of formulations including formulations to be administered during or after exercise, as well as formulations directed to enhancing sleep quality and post-workout recovery, and to reducing stress and anxiety.

To create a pre-workout supplement suitable for administering via the pulmonary route instead of the conventional oral route, first, an appropriate formulation of candidate ingredients is selected. The candidate ingredients are selected based on the desired aggregate effect of the completed formulation. While individual ingredients are known to provide certain fitness benefits, in the formulation of this embodiment candidate ingredients are selected in part based on their ability to work synergistically to provide a boost in workout performance. (Step 200)

In designing this formulation, the solubility and compatibility of each candidate ingredient are tested. The solubility of each candidate ingredient in the e-liquid base, which may be vegetable glycerin (VG) derived from coconut or palm oils or the aforementioned propylene glycol (PG), is tested and any ingredient that precipitates in the e-liquid is rejected. (Step 210) Next, to test the compatibility of each candidate ingredient with the heating conditions in a vape pen, each ingredient is heated by a vape pen vaporizer/atomizer and the ability of molecules of that candidate ingredient to withstand that heating process is evaluated. Additionally, gas chromatography (GC) or gas chromatography-mass spectrometry (GC-MS) is used to test the conservation of the functional group of individual molecules before and after the process of aerosolization to ensure proper delivery and preservation of all the intended supplement ingredients in their active form. A passing result is obtained when the resulting gas chromatogram of the tested formulation display the same specific retention time and intensity of signature functional peaks as that of an expected control (a single ingredient sample as a positive control). Alternatively, a mass spectrum obtained by GC-MS may be compared with a GC-MS library to validate the functional structures of the intended compound and to identify all the expected peaks and ensure that no abnormal peak arises. (Step 220)

A combination of vape-compatible (aerosolizable) ingredients is then carefully selected in the proper dosage ratio in the end formulation to provide the unique synergistic pre-workout benefits intended. In this step, the dosage is determined by translating a known dosage level as used in existing formulations of powdered supplements for oral administrating to a suitable dosage for pulmonary administration. The suitable dosage for pulmonary administration is calculated based on a pre-determined conversion ratio derived from the inventors' empirical analysis comparing alertness levels resulting from caffeine intake via oral administration versus pulmonary administration. (Step 230)

A standard 510 thread vape-cartridge containing the vape-compatible pre-workout supplement formulation is then manufactured in FDA-approved vape manufacturing facilities with stringent quality control. (Step 240)

The aerosolized vape is then tested to ensure no unwanted by-products are created during the heating process and then a recommended dosage level is determined based on the assumed use of a standard vape pen, with a power output of 3-5 W and an output voltage of 3V or less, and a standard vape cartridges having a resistance of around 1.5-1.65 ohms. The absence of unwanted by-products is ensured by performing gas chromatography or gas chromatography-mass spectrometry to check for the presence of peaks that reflect the production of any by-products that may have been created in the heating process. The recommended dosage level is then determined based on repeated timed inhalations of equal duration to set a threshold assumption (level below which no effects are perceived) of the dosage when pulmonarily delivered, and the recommended dosage level is set a level above this threshold. Alternatively, a recommended dosage level may be determined based on the detected bioavailability of certain desired active ingredients detected in metabolite blood analyses performed on test subject athletes. (Step 250)

The resulting end product is a standard vape cartridge containing the designed pre-workout formulation that readily aerosolizes and that is compatible in use with any standard vape pen with the 510-thread cartridge and power specification as described.

This method, as disclosed, provides at least the following advantages:

(1) use of a conventional vaporizer or atomizer to aerosolize sports supplement ingredients for pulmonary administration without the need for special equipment;
(2) systematic procedure for identifying ingredients that are compatible with the process of pulmonary delivery as described;
(3) use of a combination of multiple ingredients (the ingredients combination and their relative composition (relative ratio by composition) in the end product) to produce the sports performance enhancing effect; and
(4) application of pulmonary delivery of sports supplements to subjects prior to and during exercise/training.

The method disclosed above provides for the pre-workout pulmonary administration of athletic or fitness supplements supplement, but in other embodiments, the supplement may be pulmonarily administrated during or after a workout or other physical activity. For example, a performance-enhancing supplement may be administered during a workout or a recovery vape formulation for post-training recovery may be administered after a strenuous workout to reduce muscle pain and facility rejuvenation of fatigued muscles.

Although the embodiments above have been directed to human consumption and use, this disclosure is not limited thereto and may also be applicable to the pulmonary administration of performance-enhancing supplements to other mammals such as horses and dogs, and to the method of production of such supplements.

While the formulations and methods disclosed herein have been described in terms of what are presently considered to be the most practical and preferred embodiments, it is to be understood that the disclosure need not be limited to the disclosed embodiments. The disclosure is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of modifications may be made without departing from the essence of the disclosure and claims. Such modifications are implicitly included in the description and fall within the scope of this disclosure. It should be understood that this disclosure is intended to yield a patent covering numerous aspects of the invention both independently and as an overall system and in both method and apparatus modes.

For example, in the embodiments described above, the e-liquid cartridge and atomizer are separate components, however in other embodiments the cartridge and atomizer may be integrated together as a “cartomizer”.

Moreover, additional supplement ingredients may also be added to the formulations to provide a desired flavor or a nutritional vitamin.

What is claimed is:

1. A therapeutic composition intended for administration before, during, or after exercise, comprising:
an aerosolizable solution that includes a base liquid and a multi-ingredient mixture of supplements dissolved in said base liquid.

2. The composition of claim 1, wherein the base liquid is an e-liquid for use in an e-cigarette and the composition is housed in an e-cigarette cartridge.

3. The composition of claim 2, wherein
the base liquid comprises vegetable glycerin or propylene glycol; and
the multi-ingredient mixture of supplements is selected from the group consisting of at least three of the following ingredients: pineapple lime extract, riboflavin, L-tyrosine, L-theanine, taurine, caffeine, agmatine sulfate, citrulline malate, and beetroot.

4. The composition of claim 3, wherein
the base liquid comprises vegetable glycerin (45-99% by volume) or propylene glycol (40-99% by volume); and
the multi-ingredient mixture of supplements is selected from the group consisting of at least three of the following ingredients in the volume percentage ranges of: pineapple lime extract (0.5-3%), riboflavin (0.05-3%), L-tyrosine (0.1-0.6%), L-theanine (0.1-0.6%), taurine (0.3-1.2%), caffeine (0.3-1%), agmatine sulfate (0.05-0.3%), citrulline malate (0.1-0.6%), and beetroot (0.05-0.3%).

5. The composition of claim 4, wherein
the base liquid comprises vegetable glycerin (preferably 50% by volume) or propylene glycol (preferably 46.28% by volume); and
the multi-ingredient mixture of supplements is selected from the group consisting of at least three of the following ingredients in the preferred volume percentages of: pineapple lime extract (1%), riboflavin (0.09%), L-tyrosine (0.19%), L-theanine (0.19%), taurine (0.47%), caffeine (0.38%), agmatine sulfate (0.09%), citrulline malate (0.19%), and beetroot (0.09%).

6. The composition of claim 2, wherein
the base liquid comprises vegetable glycerin or propylene glycol; and
the multi-ingredient mixture of supplements is selected from the group consisting of at least three of the following ingredients: 5HTP, L-theanine, melatonin, rutaecarpine, gamma aminobutyric acid (GABA), chamomile flower extract, tryptophan, huperzine A, evodiamine, L-ornithine, tart cherry extract, valerian root, lavender extract, passion flower, Mucuna pruriens L-dopa, phosphatidylserine, and reishi/lingzhi.

7. The composition of claim 6, wherein
the base liquid comprises vegetable glycerin (45-99% by volume) or propylene glycol (40-99% by volume); and
the multi-ingredient mixture of supplements is selected from the group consisting of at least three of the following ingredients in the volume percentage ranges of: 5HTP (0.2-1%), L-theanine (0.25-1.5%), melatonin (0.005-0.05%), rutaecarpine (0.1-0.6%), gamma aminobutyric acid (GABA) (0.2-1.2%), chamomile flower extract (0.4-2.4%), tryptophan (1-6%), huperzine A (0.15-1%), evodiamine (0.005-0.05%), L-ornithine (0.4-2.5%), tart cherry extract (0.4-2.5%), valerian root (0.85-5.5%), lavender extract (0.15-1%), passion flower (0.4-2.4%), Mucuna pruriens L-dopa (0.5-3%), phosphatidylserine (0.05-0.3%), and reishi/lingzhi (0.1-0.5%).

8. The composition of claim 7, wherein
the base liquid comprises vegetable glycerin (preferably 50% by volume) or propylene glycol (preferably 46.28% by volume); and
the multi-ingredient mixture of supplements is selected from the group consisting of at least three of the following ingredients in the preferred volume percentages of: 5HTP (0.38%), L-theanine (0.57%), melatonin (0.01%), rutaecarpine (0.19%), gamma aminobutyric acid (GABA) (0.38%), chamomile flower extract (0.76%), tryptophan (1.9%), huperzine A (0.29%), evodiamine (0.01%), L-ornithine (0.76%), tart cherry extract (0.76%), valerian root (1.71%), lavender extract (0.3%), passion flower (0.76), Mucuna pruriens L-dopa (0.95%), phosphatidylserine (0.095%), and reishi/lingzhi (0.15%).

9. The composition of claim 2, wherein
the base liquid comprises vegetable glycerin or propylene glycol; and
the multi-ingredient mixture of supplements is selected from the group consisting of at least three of the following ingredients: gamma aminobutyric acid (GABA), chamomile flower extract, passion flower, bacopa monnieri, L-phenylalanine, L-theanine, lemon balm, gotu kola, aniracetam, valerian root, phenibut, alpha-GPC, ashwagandha, Rhodiola rosea, PQQ, noopept, cat's claw, and oat straw.

10. The composition of claim 9, wherein
the base liquid comprises vegetable glycerin (45-99% by volume) or propylene glycol (40-99% by volume); and
the multi-ingredient mixture of supplements is selected from the group consisting of at least three of the following ingredients in the volume percentage ranges of: gamma aminobutyric acid (GABA) (0.2-1.2%), chamomile flower extract (0.4-2.4%), passion flower (0.4-2.4%), bacopa monnieri (0.25-1.5%), L-phenylalanine (0.2-1.2%), L-theanine (0.25-1.5%), lemon balm (0.25-1.5%), gotu kola (0.5-3%), aniracetam (0.1-0.6%), valerian root (1-6%), phenibut (1-6%), alpha-GPC (1.1-7%), ashwagandha (0.4-2.4%), Rhodiola rosea (0.5-3.5%), PQQ (0.02-0.12%), noopept (0.015-0.1%), cat's claw (0.5-3%), and oat straw (1.1-7%).

11. The composition of claim 10, wherein
the base liquid comprises vegetable glycerin (preferably 50% by volume) or propylene glycol (preferably 46.28% by volume); and
the multi-ingredient mixture of supplements is selected from the group consisting of at least three of the following ingredients in the preferred volume percentages of: gamma aminobutyric acid (GABA) (0.38%), chamomile flower extract (0.76%), passion flower (0.76%), bacopa monnieri (0.57%), L-phenylalanine (0.38%), L-theanine (0.57%), lemon balm (0.57%), gotu kola (0.95%), aniracetam (0.19%), valerian root (1.9%), phenibut (1.9%), alpha-GPC (2.28%), ashwagandha (0.76%), Rhodiola rosea (1.14%), PQQ (0.038%), noopept (0.029%), cat's claw (0.95%), and oat straw (2.28%).

12. The composition of claim 2, wherein
the base liquid comprises vegetable glycerin (45-99% by volume) or propylene glycol (40-99% by volume); and
the multi-ingredient mixture of supplements is selected from the group consisting of one or more of the following ingredients: tart cherry extract, L-carnitine, 5-HTP, L-theanine, melatonin, magnesium, zinc, L-glycine, rutaecarpine, gamma aminobutyric acid (GABA), CBD, chamomile flower extract (Matricaria recutita), lavender extract, passion flower (Passiflora incarnata), bacopa monnieri, lemon balm, tryptophan, huperzine A, gotu kola, aniracetam, quercetin, tribulus terrestris, vitamin C, vitamin E, coenzyme Q10, dehydroepiandrosterone (DHEA), curcumin, palmitoylethanolamide, Mucuna pruriens L-dopa, L-phenylalanine, L-ornithine, valerian root, phenibut, alpha-GPC, ashwagandha (KSM-66), evodiamine, phosphatidylserine, DMAE, hops strobile, lemon balm leaf, bacopa monnieri, Rhodiola rosea, sulbutiamine, alpha-lipoic acid, choline, PQQ, vinpocetine, pine bark, reishi/lingzhi, cordyceps, lion's mane, chaga, shiitake, pterostilbene, resveratrol, uridine monophosphate, theobromine, ginko biloba leaf, sulbutiamine, noopept, cat's claw, oat straw, L-norvaline, hordenine, synepherine, alcar, theobromine, elderberry, and zinc.

13. The composition of claim 12, wherein the base liquid comprises vegetable glycerin (preferably 50% by volume) or propylene glycol (preferably 46.28% by volume).

14. A method for producing the composition of claim 2 comprising the steps of:
selecting the base e-liquid and the supplement ingredients;
testing the solubility of each supplement ingredient in the base e-liquid;
testing the compatibility of each of the supplement ingredients and the base e-liquid with aerosolization by a vape pen;
selecting a combination of soluble and vape pen compatible ingredients to provide synergistic benefits and dissolving said combination in the base e-liquid to create a vape-compatible formulation;
injecting the vape-compatible formulation into a standard vape cartridge; and
determining a recommended dosage of the vape-compatible formulation in the vape cartridge.

15. A method for improving athletic performance in mammals, comprising the steps of:
pulmonarily administering a therapeutic dose of a supplement to the mammal prior to athletic activity; and
controlling the timing and dosage of the administered supplement.

16. A method for improving athletic performance in mammals, comprising the steps of:
pulmonarily administering the composition of claim 3 to the mammal prior to athletic activity; and
controlling the timing and dosage of the administered supplement.

17. The method of claim 15, further comprising the step of aerosolizing by heating the therapeutic dose of the supplement prior to pulmonarily administering said supplement.

18. The method of claim 16, further comprising the step of aerosolizing by heating the composition prior to pulmonarily administering said composition.
